(19) United States
(12) Patent Application Publication
(10) Pub. No.: US 2005/0159798 A1
(43) Pub. Date: Jul. 21, 2005

(54) Method and apparatus for cardiac ablation

(76) Inventors: Rainer Graumann, Hochstadt (DE); Norbert Rahn, Forchheim (DE)

Correspondence Address: SCHIFF HARDIN LLP;Patent Department, 6600 Sears Tower, 233 South Wacker Drive, Chicago, IL, 60606, US

(21) Appl. No.: 10/983,147

(22) Filed: Nov. 5, 2004

(30) Foreign Application Priority Data
Nov. 7, 2003 (DE) 103 52 011.2

(51) Int. Cl. A61F 2/00

(52) U.S. Cl. 607/101

(57) ABSTRACT

In an apparatus and method for cardiological ablation an endoscope with an integrated optical camera and an instrument access channel is guided in a patient by an endoscope control and processing device and an endoscope visualization device, and an RF ablation wire is actively navigated by means of a magnetic navigation system into the instrument access channel.

### BACKGROUND OF THE INVENTION

1. Field of the Invention

The present invention in general concerns ablation techniques in cardiology. The present invention in particular concerns a system and method for improved ablation in the context of a pulmonary vein isolation.

2. Description of the Prior Art

How fast the human heart beats is dependent on nerve impulses that the sinus node sino-atrial node, at the right atrium of the heart emits, 60 to 80 heartbeats a minute are regarded as normal. This normal rhythm can be disturbed; the trigger of heart rhythm disturbances is individually very different. A possible trigger is, for example, a pathological interference of the electrophysiological heart conduction system.

If due to formation of pathological excitation centers or heart conduction paths, there are other impulse emitters in the atrium or chamber of the heart, the actual pacemaker (sinus node) can be deactivated and the normal excitation conduction can be falsely replaced by uncoordinated impulses. When the excitation formation is diffuse and ensues at different points, in the worst case a “normal” heartbeat pulse cannot propagate at all. The monitored heart muscle movement is in this case very severely disturbed; the heart chambers only “flutter” or “flicker”. This represents a life-threatening situation that must be controlled immediately via electroshock treatment or medication measures, otherwise cardiac arrest occurs.

If electroshock treatment and/or medication treatment is not successful, or the therapy is not or administered at sufficient levels, conductor paths that are responsible for pathological pulse conduction or pulse formation can be interrupted by means of heart catheter techniques (in rare cases also classical surgery). The elimination of pathological excitation centers or heart conduction paths in the heart is presently implemented primarily by RF ablation (radio-frequency ablation). For this, a catheter is inserted into the body in a typical manner via a large body vein or artery and is guided into the affected heart chamber. Radio-frequency electromagnetic energy is applied to the site via an electrode located at the catheter tip in order to obliterate affected tissue and thus to interrupt the pathological excitation centers or heart conduction paths.

An ablation procedure of the type just described is specifically executed given atrial flickering or atrial fluttering.

Causes for an atrial flicker can be pathological heart conduction paths that propagate in the form of myocardial fibers from the (in total) four pulmonary veins to the left atrium. The oxygen-enriched blood of the lungs is again supplied to the heart via the pulmonary veins. The cause for the formation of such heart conduction paths is still unclear. Such heart conduction paths lead to the atrium contraction being falsely triggered (sometimes with a frequency of over 200 contraction cycles per minute).

According to the prior art, an ablative therapy or an ablation in the context of a sustained therapy regimen occurs in a technique known as pulmonary vein isolation, in which the four pulmonary veins are electrophysiologically separated from the left atrium. This occurs by circular or annular RF ablation in the ostium (opening region) of the pulmonary veins (tantamount to the creation of an annular lesion).

In specific cases of atrial flickering, a linear lesion is additionally, generated by an RF ablation by means of catheter. This proceeds along an imaginary connection line of the four pulmonary veins with the mitral valve (the heart valve between the heart chamber and the left atrium).

The guidance of a heart catheter 11 given transseptal examination, or ablation is schematically shown in FIG. 2. The heart and the blood-supplying and blood-discharging vessels are significantly simplified and shown in cross-section. The upper half of the heart includes the right atrium 15 and the left atrium 14, while the lower part is formed by the right heart chamber 17 and the left heart chamber 16. The atria 14, 15 and the heart chambers 16, 17 are separated by a septum. The oxygen-poor blood is supplied to the right atrium 15 via the superior vena cava 20 as well as via the inferior vena cava 21 and is pumped from the right heart chamber 17 into the lungs via the aorta pulmonaris 18. The pressure necessary for this is generated by controlled (sinus node-controlled), periodic contraction of the heart muscle. The oxygen-enriched blood in the lungs arriving into the left atrium 14 via the pulmonary veins 13 (only two of four are illustrated in FIG. 2) is directed into the left heart chamber and forced from the left heart chamber 16 into the entire body (the arrows symbolize the direction of the blood flow) via the aorta 19.

In order to implement catheter-based pulmonary vein isolation, the catheter is preferably inserted into the superior vena cava 21 in the inguinal region groin and is advanced into the right atrium 15.

In order to reach the ostium of the pulmonary veins 13, it is subsequently necessary to pierce the septum 22 and to insert the catheter Into the opening regions of the pulmonary vein. Circular or annular lesions that electrophysiologically sever the myocardium phases of the pulmonary veins from the heart tissue are produced by the application of RF energy in the ostium of the pulmonary veins 13 from an electrode at the catheter tip 12. In a possible further variant of the ablation, linear lesions are produced which effectively electrophysiologically rectangularly confine all four pulmonary veins from the surrounding tissue of the left atrium.

Problems associated with this application or with the use of a catheter-based ablation system are as follows:

Due to the limited freedom of movement of a catheter, it is very difficult and time-consuming to direct the catheter into the left atrium and then into a pulmonary vein and to subsequently with precision ablate at that site, the more so since the feed ensues into the left atrium via a transseptal penetration (puncture of the atrium partition wall) from the right atrium. Additionally, during the ablation the guidance of a catheter exactly into the ostium of the pulmonary veins is markedly difficult because the ostium typically is insufficiently clearly shown in conventional intra-operative x-ray images (for example with a C-arm x-ray imaging system, the presently most common visualization method) and moreover it is only two-dimensionally imaged.

However, if the RF ablation does not occur exactly in the ostium, but rather (for example) more distal in the pulmonary vein the danger of stenotization (narrowing or sealing) the pulmonary vein exists, which requires additional procedures such as the placement of a stent in the vein. In general, in an RF ablation the danger exists of the tissue overheating, which in particular can lead to blood clots (thrombosis) inside the blood vessels and thus to a heart attack.

The already-mentioned placement of linear lesions at the mitral valve is also complicated (the procedure lasts up to 9 hours). The success is dependent on the continuity of the generated lesion lines, which are generated point-by-point, and the continuity can only be checked adequately only with x-ray radioscopy. Ultimately, the pulmonary vein isolation implemented with a catheter exhibits overall a success rate of 00 to 70%, which is not acceptable given therapeutic effort and the associated risk for the patient.

In order to increase the success rate, it is presently attempted to use alternative ablation techniques to RF ablation, for example cryo-ablations (obliteration by bold) or HIFU ablations (High Focused Ultrasound, obliteration by highly-focused ultrasound). In this context, it is likewise attempted to use additional imaging systems such as, for example, intra-cardial ultrasound, preoperative CT imaging, or preoperative MRT imaging in order to be able to have an optimally large amount of information available for the ablation.

The quality of a placed lesion is dependent on the degree of its continuity and is already verified during the procedure. The test ensues with electrophysiological mapping systems (for example carto-systems by the company Biosense Webster Ltd., Diamondbar, USA) in which the organ region of interest is scanned based on EKG. This leads to the aforementioned success rates of pulmonary vein isolations in the range of 60 to 70% which, as already emphasized, is much too low relative to the operative effort and risk.

### SUMMARY

It is an object of the present invention to provide a system that enables a less-complicated cardiological ablation and overall leads to higher success rates.

This object is achieved according to the invention by an apparatus for cardiological ablation having an endoscope with an integrated optical camera and an instrument access channel, an endoscope control and processing device, as well as an endoscope visualization device, wherein an RF ablation wire can be actively navigated in the instrument access channel by a magnetic navigation system.

The above object also is achieved in accordance with the invention by an apparatus for cardiological ablation having n endoscope with an integrated optical camera and an instrument access channel, an endoscope control and processing device, as well as an endoscope visualization device, wherein an RF ablation wire is guided in the instrument access channel, the RF ablation wire being produced from a shape memory alloy and being able to be heated by the endoscope control and processing device such that, upon reaching a critical temperature, it deforms into at least one loop that corresponds to the diameter of a blood vessel to be obliterated.

In a further embodiment of the present invention, the wall contact of the RF ablation wire with the tissue to be obliterated can be advantageously, electrophysiologically measured via the RF ablation wire.

The present invention is particularly advantageous when the blood vessel to be obliterated is a pulmonary vein.

The above object also is achieved in accordance with the invention by a medical procedure for improved cardiological ablation wherein, under visual inspection of a minimally-invasively inserted endoscope, the physician places an RF ablation wire (inserted via the instrument access channel of the endoscope) at a blood vessel to be obliterated.

In a first embodiment of the inventive procedure, the placement ensues by active magnetic navigation, by magnetic fields surrounding the patient being varied by a joystick or computer mouse.

In a second embodiment of the inventive procedure, the placement ensues by memory effect, by the RF ablation wire produced from a shape memory alloy being heated by the endoscope control and processing device until, upon reaching a critical temperature, the wire deforms into at least one loop that corresponds to the diameter of the blood vessel to be obliterated.

In an embodiment of the inventive procedure, it is possible that the wall contact of the RF ablation wire with the tissue to be obliterated is electrophysiologically measured by the RF ablation wire.

In the scope of the medical procedure, the blood vessel can be a pulmonary vein.

### DESCRIPTION OF THE DRAWINGS

FIG. 1 is a schematic perspective representation of the human heart with an endoscope minimally-invasively administered up to the ostium of a pulmonary vein, in inventive combination with a magnetic navigation system.

FIG. 2 is a schematic perspective representation of the human heart with endoscope minimally-invasively administered up to the ostium of a pulmonary vein, in inventive combination with an RF ablation wire produced from a shape memory alloy.

FIG. 3 schematically shows the heart in cross-section during heart catheterization upon transseptal examination or ablation.

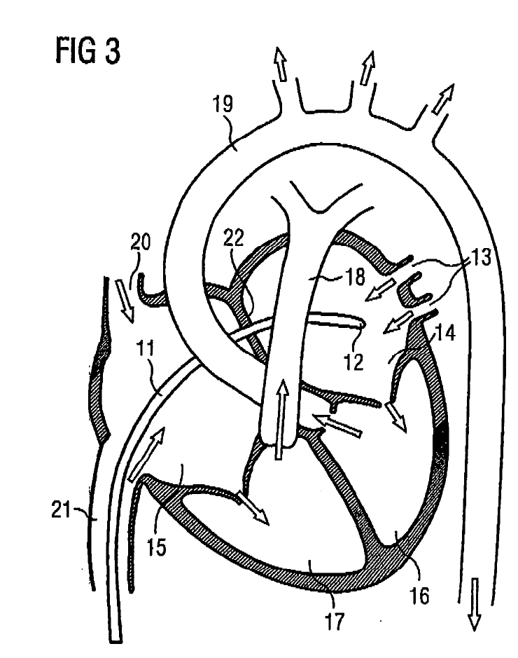

### DESCRIPTION OF THE PREFERRED EMBODIMENTS

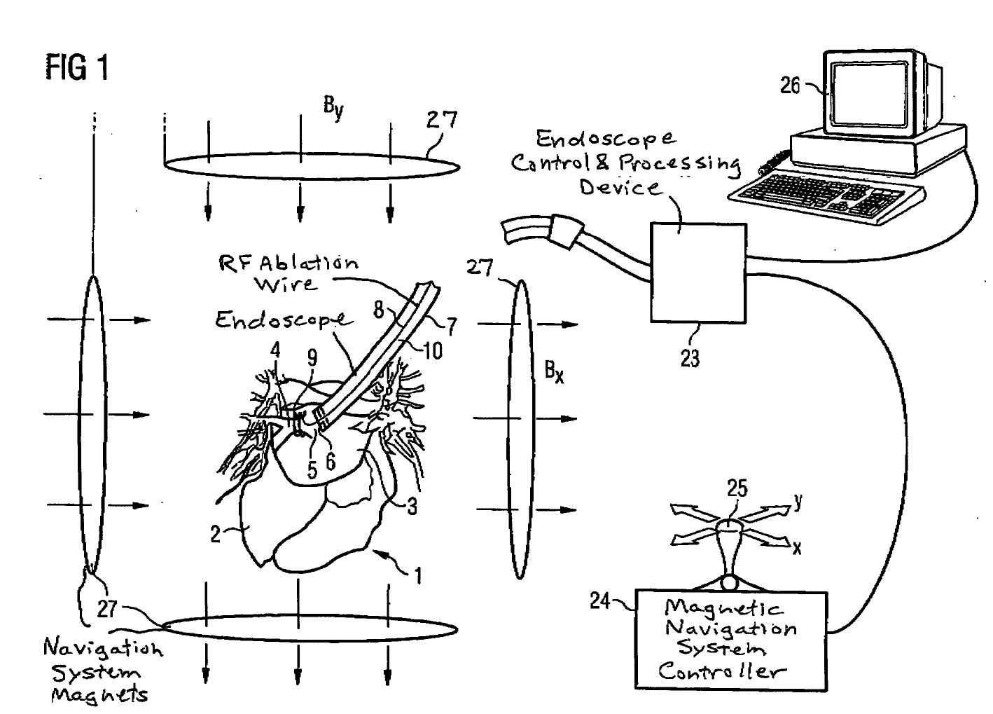

FIG. 1 shows the inventive apparatus in a perspective representation of the externally endoscoped heart with a magnetic navigation system. The left heart chamber 2, to which the left atrium 3 connects at the top, is visible in the lower region. The left atrium in turn shows one of the four existing pulmonary veins 4 that discharge into the ostium 5 in the left atrium 5 and are isolated in an efficient manner in precisely this region with the inventive apparatus. The inventive apparatus according to FIG. 1 is formed by the combination of variable magnetic fields with an endoscope 7 and a magnetic RF ablation wire 8 pushed through the endoscope tip 6 via the instrument access channel 10. Via the RF ablation wire 8 either RF radiation can be applied on tissue to be coagulated (obliterated) or the end or tip thereof can be heated such that an obliteration ensues with the wire tip by heating of the tissue. The RF ablation wire or its magnetic end 9 can be actively navigated with the aid of spatially-variable magnets or magnetic fields. Such a navigation system is, for example, produced and distributed by the company Stereotaxis, St. Louis, USA under the model designation “Niobe”. The navigation ensues under visual inspection of the endoscope, which typically has at its tip integrated optics 6 (illumination, camera). Additional further techniques such as, for example, intra-cardial ultrasound and/or (C-arm) x-ray radioscopy can be used use for imaging. The magnetic fields (Bx, By, etc.) are generated either by coil magnets 27 or permanent magnets, which are arranged around the patient. A variation of the magnetic fields effects a direction change or a corresponding alignment of the magnetic RF ablation wire and, for example, ensues by means of joystick 25 (or computer mouse) which represents a part of the controller of the magnetic navigation system 24. The endoscope 7 can itself in particular be navigated in the end region (endoscope tip 6, region with integrated optics 6) via the control and processing device 23; the endoscope images are shown in the screen of the endoscope visualization unit 26.

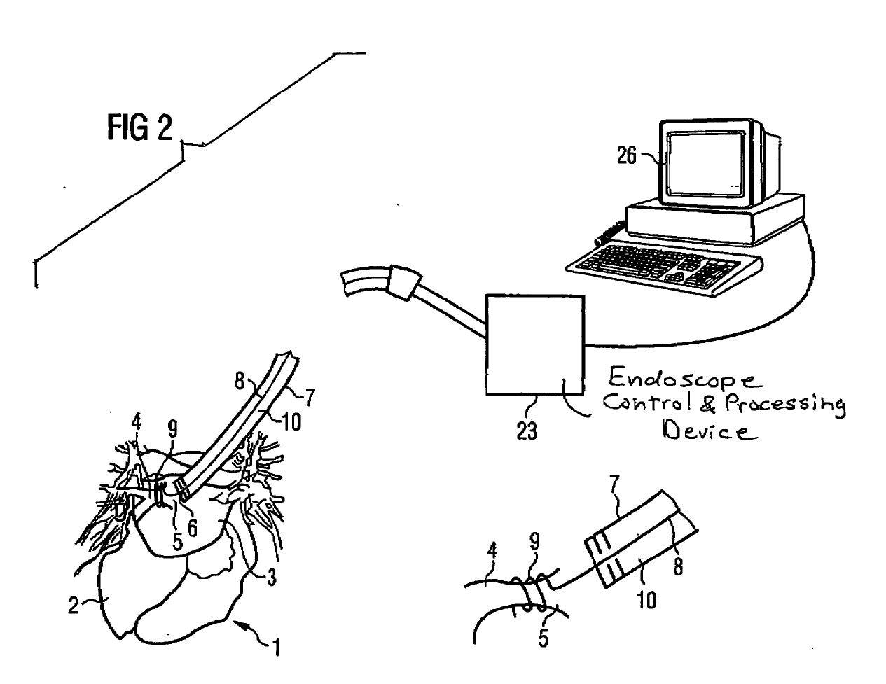

Another possibility for the precise placement of the end section 9 of the RF ablation wire is to produce the end section from a shape memory alloy (memory metal), namely such that the wire end section 9 wraps around the pulmonary vein to be ablated upon reaching a critical temperature. The heating of the RF ablation wire or, respectively, its end section ensues via the endoscope control and processing device. In FIG. 2, for example, two loops are shown. A further heating of the RF ablation wire end section 9 or, respectively, an application of RF radiation finally effects a coagulation of the looped blood vessel in the region of the looped locations.

Due to both of these advantageous, possibly combined properties of the wire or the wire tip, the RF ablation wire can be inventively, exactly placed at the location (especially at the ostium 5) to be scored.

Moreover, it is a further aspect of the present invention to be able to monitor or measure the wall contact of the RF ablation wire before and during the point in time of the ablation via the tip or via the end region of the radio-frequency ablation wire by a suitable technique electrically similar to the already-mentioned mapping system, in order to be able to assess the quality of the set lesion.

An RF ablation wire with the cited properties in combination with an endoscope represents an inventive apparatus, which significantly eases a pulmonary vein isolation for the user. Instead of being directed with the assistance of an intra-cardially guided catheter, the endoscope is minimally-invasively directed through the chest or through the back of the patient and is guided under visual inspection via the endoscope optics 6 to the anatomical position to be ablated.

The RF ablation wire is supplied through the endoscope access channel to the pulmonary vein or its ostium under optical visual inspection and/or electrically-measured wall contact, and can thus be placed exactly at the point to be obliterated. The ablation thus ensues at the outer wall of the pulmonary vein or given linear lesions, at the outer wall of the left atrium.

This inventive apparatus and the inventive procedure has numerous advantages:

The endoscope can be freely directed in the body to the pulmonary vein to be isolated, A complicated navigation through the heart with transseptal penetration is no longer necessary. The ablation no longer occurs in the lumen (meaning in the region of the vein supplied with blood). The risk of the blood clotting (thrombosis), which can lead to a heart attack, does not exist. Under visual inspection and electrical verification of the wall contact of the RF ablation wire, the lesions, in particular linear lesions, can ensue more precisely and efficiently. The endoscope-based visual inspection provides additional anatomical information an improves the placement of the RF ablation wire in, the region of the pulmonary vein ostium. This leads to a significant increase of the success rate of the pulmonary vein isolation. By ablation on the outer wall of the veins, it is assumed that the risk of the vein stenotization is substantially reduced.

Although modifications and changes may be suggested by those skilled in the art, it is the intention of the inventors to embody within the patent warranted hereon all changes and modifications as reasonably and properly come within the scope of their contribution to the art.

1. An apparatus for cardiac ablation comprising:
an endoscope having an optical camera integrated therein and containing an instrument access channel;
an endoscope control and processing device connected to said endoscope for operating said endoscope;
a visualization device connected to said control and processing device for displaying an image obtained with said optical camera;
an RF ablation wire disposed in and movable in said access channel; and
a magnetic navigation system that interacts with said RF ablation wire for guiding said RF ablation wire in said instrument access channel.

2. An apparatus as claimed in claim 1 wherein said control and processing device allows a measurement of a degree of contact of said RF ablation wire with tissue to be ablated.

3. An apparatus as claimed in claim 1 wherein said RF ablation wire is adapted for ablating tissue in a pulmonary vein.

4. An apparatus for cardiac ablation comprising:
an endoscope having an optical camera integrated therein and containing an instrument access channel;
an endoscope control and processing device connected to said endoscope for operating said endoscope;
a visualization device connected to said control and processing device for displaying an image obtained with said optical camera; and
an RF ablation wire disposed in and movable in said access channel, said RF ablation wire being composed of a shape memory alloy heatable by said control and processing device for, upon reaching a critical temperature, deforming into a loop corresponding to a diameter of a blood vessel to be ablated.

5. An apparatus as claimed In claim 4 wherein said control and processing device allows a measurement of a degree of contact of said RF ablation wire with tissue to be ablated.

6. An apparatus as claimed in claim 4 wherein said RF ablation wire is adapted for ablating tissue in a pulmonary vein.

7. A method for cardiac ablation comprising the steps of:
providing an endoscope with an instrument access channel;
introducing the endoscope into a blood vessel containing tissue to be ablated;
inserting an RF ablation wire into the instrument access channel of the endoscope; and
visually guiding said RF ablation wire relative to said tissue using an image of the tissue obtained with the endoscope.

8. A method as claimed in claim 7 comprising guiding said RF ablation wire in said instrument access channel with an input unit of a magnetic navigation system.

9. A method as claimed in claim 7 comprising producing said RF ablation wire, a shape memory alloy, and heating said RF ablation wire in said instrument access channel for causing said RF ablation wire, upon reaching a critical temperature, to deform into a loop corresponding to a diameter of said blood vessel at a location of said tissue.

10. A method as claimed in claim 7 comprising monitoring contact of said RF ablation wire with said tissue via said RF ablation wire.

11. A method as claimed in claim 7 comprising introducing said endoscope and said RF ablation wire into a pulmonary vein as said blood vessel.
